(19) United States
(12) Patent Application Publication
(10) Pub. No.: US 2025/0322933 A1
(43) Pub. Date: Oct. 16, 2025

(54) ARTIFICIALLY INTELLIGENT BATHROOM

(71) Applicant: Deep Detection LLC, Troy, NY (US)

(72) Inventors: Alexander Bringsjord, Rensselaer, NY (US); Harshil Jain, Troy, NY (US); Hannah Kittrell, Troy, NY (US)

(21) Appl. No.: 19/179,306

(22) Filed: Apr. 15, 2025

Related U.S. Application Data
(60) Provisional application No. 63/634,021, filed on Apr. 15, 2024.

(51) Int. Cl. G16H 20/60; A61B 10/00; E03D 9/00; G16H 50/30

(52) U.S. Cl. CPC G16H 20/60; A61B 10/0038; E03D 9/00; G16H 50/30

(57) ABSTRACT

An intelligent toilet system and method. The system includes a toilet having a waste analysis system, where the waste analysis system includes sensors that analyze microbiome in human waste of a user; an artificial intelligence (AI) system that receives sensor data from the waste analysis system and generates a health profile of the user; and a hypergraphical platform that generates a dietary plan from the health profile and validates the plan using logics.

### PRIORITY

This application claims benefit to co-pending provisional application filed on Apr. 15, 2024, Ser. No. 63/634,021, entitled Intelligent Toilet, the contents of which are hereby incorporated by reference.

### BACKGROUND

### 1. Technical Field

This invention relates generally to an intelligent toilet and bathroom that utilizes artificial intelligence and a logics-based planning and validation system.

### 2. Related Art

Gut health is intricate to overall health, and can be improved through changes in lifestyle. But, in order to make these changes, we need to be able to detect problems even when we cannot see them. There exist various smart toilets that for example include sensors to monitor health.

### SUMMARY

The present invention provides an intelligent toilet system and method.

In a first aspect, the invention provides an intelligent toilet system, comprising: a toilet having a waste analysis system, where the waste analysis system includes sensors that analyze microbiome in human waste of a user; an artificial intelligence (AI) system that receives sensor data from the waste analysis system and generates a health profile of the user; and a hypergraphical platform that generates a dietary plan from the health profile and validates the plan using logics.

In a second aspect, the invention provides a method, comprising: providing a toilet having a waste analysis system; collecting human waste of a user in the waste analysis system; analyzing microbiome in the human waste and generating sensor data; using an artificial intelligence (AI) system to process the sensor data and generate a health profile of the user; and generating a dietary plan from the health profile and validating the dietary plan using a hypergraphical platform with logics.

### BRIEF DESCRIPTION OF THE DRAWINGS

FIG. 1 depicts an intelligent toilet system in accordance with an embodiment of the invention.

FIG. 2 depicts an illustrative hypergraphical GUI in accordance with an embodiment of the invention.

FIG. 3A depicts an illustrative hypergraphical GUI for a dietary plan in accordance with an embodiment of the invention.

FIG. 3B depicts an illustrative hypergraphical GUI for an EMS response in accordance with an embodiment of the invention.

FIG. 4 depicts a planning platform in accordance with an embodiment of the invention.

FIG. 5 depicts a computing system in accordance with an embodiment of the invention.

### DETAILED DESCRIPTION

The disclosed intelligent toilet system expands on existing smart toilet remote patient monitoring capabilities by combining remote patient monitoring, advanced AI, planning and validation (e.g., using automated HyperGraphical reasoning, Natural Language Processing, etc.), emergency response, and dietary analysis into a holistic system.

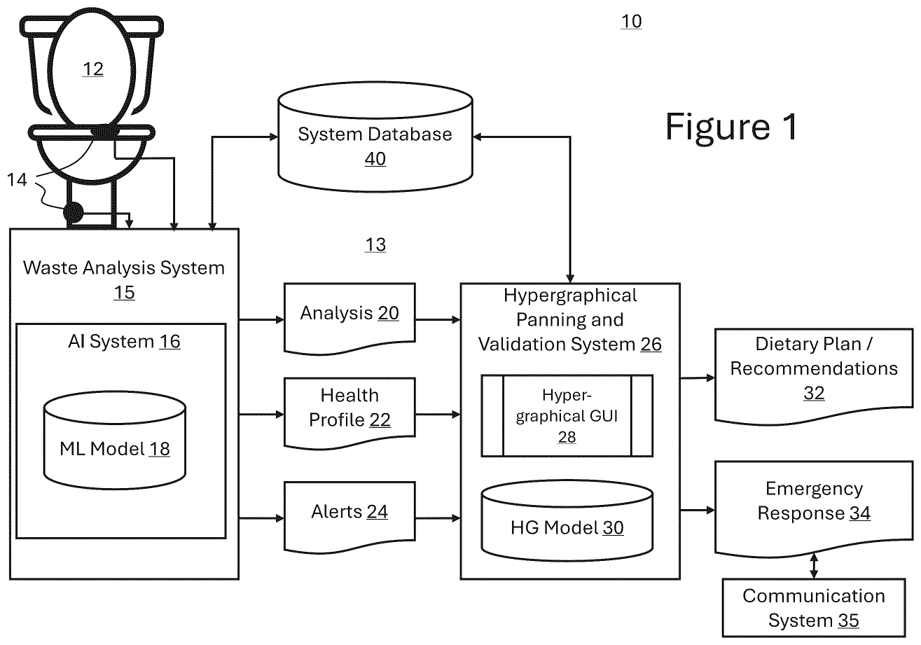

Referring now to the drawings, FIG. 1 depicts an intelligent toilet system 10 that includes a smart toilet 12 and an associated data processing architecture 13 to address gut-related diseases, personalized nutrition, and emergency/urgent medical response situations. Smart toilet 12 includes an integrated waste analysis system 15 and a set of sensors 14 for real-time monitoring of a user, which includes the ability to automatically collect sensor data associated with urine and fecal samples. The smart toilet 12 may for example include a filtration component responsible for the automated collection of biological waste directly from the toilet, ensuring seamless and hygienic sample acquisition. Once collected, waste analysis system 14 generates sensor data including microbiome information. The sensor data is stored in system database 40 and fed to artificial intelligence (AI) system 16. AI system 16 includes a trained model 18 configured to automatically detect physiological indicators associated with various health conditions in real-time. For example, systems 15 and 16 might for example generate analysis 20, a heath profile 22, and alerts 24. Following collection, the automated waste analysis component may utilize DNA sequencing technology to profile the user's gut microbiome. The sensor data may for example be cleaned and analyzed by AI system 16, ensuring precision and reliability in the results. The entire process is automated end-to-end and is executed remotely using advanced sensory technology capable of real-time processing.

One of the pivotal features of the Intelligent Toilet system 10 is its ability to detect changes in the microbiome composition. The platform enabling the Intelligent Toilet will provide as the health profile 22 physiological indicators that could be indicative of underlying health issues and changes in dietary intake. By comparing new data with an established baseline (e.g., historical health profiles), the Intelligent Toilet can identify deviations in the gut microbiome associated with dietary intake, gut-related diseases, and other health conditions.

The results from waste analysis system 15 are subsequently fed to hypergraphical (HG) planning and validation system 26. HG planning and validation system 26 includes an HG interface 28 and model 30 to generate plans (such as dietary plans 32) and validate the plans using hypergraphical reasoning (i.e., logics). Examples of hypergraphical planning and reasoning are for example disclosed in US patent application 2023/0368321 entitled ARTIFICIALLY INTELLIGENT EMERGENCY RESPONSE SYSTEM filed on May 10, 2023, and U.S. Pat. No. 11,526,779 entitled Artificial intelligence platform for auto-generating reasoning problems and assessing solutions, issued on Dec. 13, 2022, the contents of both are hereby incorporated reference.

HyperSlate® is an embodiment of a HG planning and validation system 26 capable of ingesting output from AI system 16, embedding results of that output (e.g., a statistical findings table stored in a LaTeX—output from e.g., R) within a node in a program, proof, argument, etc., within HyperSlate. The automated reasoning technology provided in HyperSlate then enables verification of the results from the AI system 16. This output can be visualized using e.g., a function that represents declarative information about the statistical results. This all enables a hybrid approach to AI: machine learning & logic-based AI, providing statistical results on the ML side, e.g., probability of gut health issues, probability of diseases, probability of a potential heart attack or probability of actual cardiac arrest (derived as disclosed from e.g., motion sensors, weights in bathroom floor/sink/etc.) integrated with HyperSlate. Then HyperSlate “tests” (i.e., verifies) the results. Verification addresses the face that in high-stakes AI environments like emergency response, probability alone may not be a sufficient long-term solution.

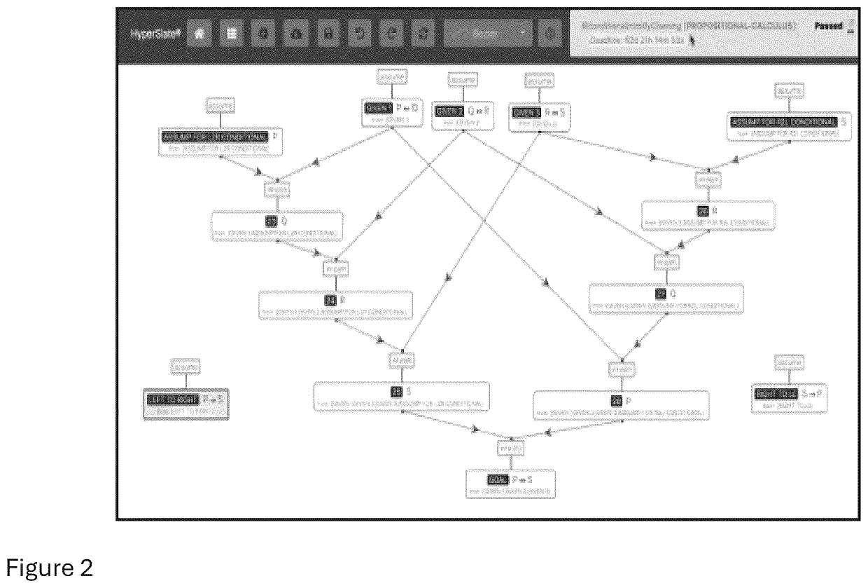

FIG. 2 depicts an example of a hypergraphical interface configured to implement real-time dietary analysis, planning, and associated recommendations for the individual requiring them. The present approach uses hybridized logic-based technology with state-of-the-art AI techniques to enable efficient and fully autonomous statistical analysis to obtain favorable probabilistic outcomes that will advise the automated planning side. Dietary recommendations, based on dietary analysis obtained from the ingesting and parsing of results from intelligent urine/fecal sensory technology are validated by logics reasoning provided by hypergraphical validation (e.g., as provided by HyperSlate). In certain approaches, the dietary analysis technology does not require analysis of fecal or urine.

The relationship between diet, the gut microbiome, and health is well-established. There are a variety of bacteria within the microbiome, and what we eat accounts for around 20% of that variation. Importantly, some bacteria are associated with improved health outcomes while others are associated with increased risk for cardiometabolic disease like diabetes and heart disease. The present approach analyzes fecal or urine samples to track shifts in microbiome population and diversity, and concentration of fecal metabolites. Microbiome diversity and composition are associated with habitual dietary intake, diet quality, and cardiometabolic risk factors. The present methods offer invaluable insights into dietary intake and gut health, and their association with an individuals' cardiometabolic risk.

The core of the dietary analysis functionality lies in AI algorithms that meticulously correlate microbiome data with known dietary factors, enabling the generation of personalized assessments and tailored dietary recommendations. The dietary analysis utilize the system database that includes an extensive array of dietary factors and their known impacts on the microbiome. By analyzing the detected microbiome composition and pairing it with the database, the present system can accurately infer dietary habits and suggest modifications to promote gut health and overall wellness. Through integration with food tracking apps, microbiome composition and diversity can also be compared to baseline data to understand the influence of dietary changes on gut health and wellness. The database is populated with an extensive array of dietary factors and their known impacts on the microbiome, and fecal and urine metabolites. The planning system supports personalized nutrition, i.e., it infers dietary intake from microbiome analysis, identifies a personal ‘baseline’ and suggests dietary changes to improve gut health, and overall wellness.

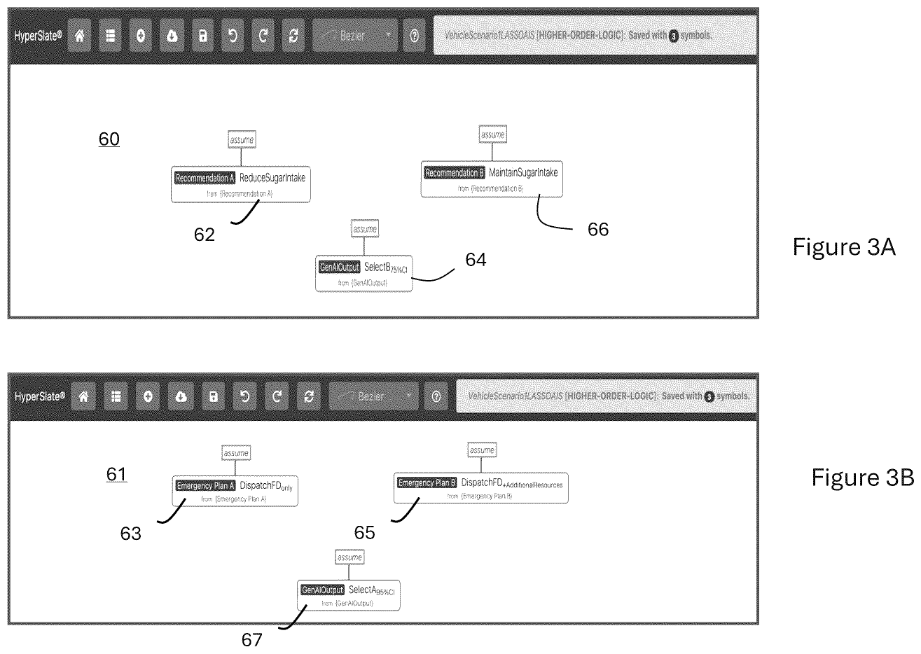

FIG. 3A depicts an example of a hypergraphical GUI 60 for generating a dietary plan. In this example, three assumptions/recommendations are for example generated by the AI system, including Reducing Sugar Intake 62, Select a B vitamin 64 and Maintain Sugar Intake. Each one of the assumptions/recommendations can be the basis of an autogenerated plan and validated using logics and reasoning. For example, the Reducing Sugar Intake 62 node can be expanded to provide a dietary plan. The plan can be validated based on the existing models and logics, e.g., as shown in FIG. 2. For example, the system can use semantic information that provides formalized knowledge of the user, e.g., user habits, behaviors, beliefs, etc., to formalize and validate a plan.

In one embodiment, the Intelligent Toilet System can detect issues in real-time to indicate a heart attack, stroke, or similar is imminent. The methods include the ability for the Intelligent Toilet to automatically notify first-responders (e.g., EMS personnel) and dispatchers that the person currently on or near the toilet is at risk of immediate death. The toilet is able to measure vitals and detect abrupt changes in body temperature, through use of sensors in the seat. The Intelligent Toilet additionally detects changes in weight, pressure, and more through all time periods of bathroom usage and uses that data to inform our logic-based AI system what to attempt to substantiate (by finding a cogent proof or argument), and then ultimately output. Therefore, the methods take not only existing filtration system(s) further, but take the conception of a toilet seat with intelligence further as well. In fact, given the weight distribution detection abilities and more, the entire toilet is an informative machine capable of harnessing all biometric data available. The seat itself will be capable of detecting bacteria that lingers from human usage, taking our claims much further than the prior art-mostly specific to monitoring vitals—for intelligent toilet seats. These bacteria-detection capabilities enable proactive prevention of diseases that have infected humans since toilets were first manufactured and put to shared use. These features combine—with other methods and components—to holistically address the problems listed herein.

The overall method combines and expands technologies, and (2) implements a dynamic ecosystem that integrates all relevant technologies and delivers a variety of outputs that are contextually dependent. The approach merges an intelligent first-responder system, a disease-prevention system, and an Intelligent Toilet with filtration, biometric, sensory, etc. The AI technology, additionally, is capable of formally justifying (and in some cases outright proving) all outputs. It can cogently justify/prove, in real-time, that the human has gastrointestinal issues, has gone into cardiac arrest, or has—in the worst case-expired. Depending on the output, FD, PD, EMS, etc. will be automatically contacted and provided a detailed explanation of the current emergency-response situation in natural language by means of Natural Language Processing (NLP). Specifically, in this context, the generated speech or text is mainly dependent on Natural Language Generation (NLG); Natural Language Understanding (NLU) kicks in upon responsive queries from the dispatcher. NLG again provides needed responses to said dispatcher queries. This process concludes upon the dispatcher aligning and contacting all needed resources.

FIG. 3B depicts a hypergraphical GUI 61 for an emergency response. In this example, three assumption/planning nodes are provided including dispatch fire department 63, dispatch fire department and additional resources 65, and generate further AI output 67. Each node can be expanded to generate and verify a final plan.

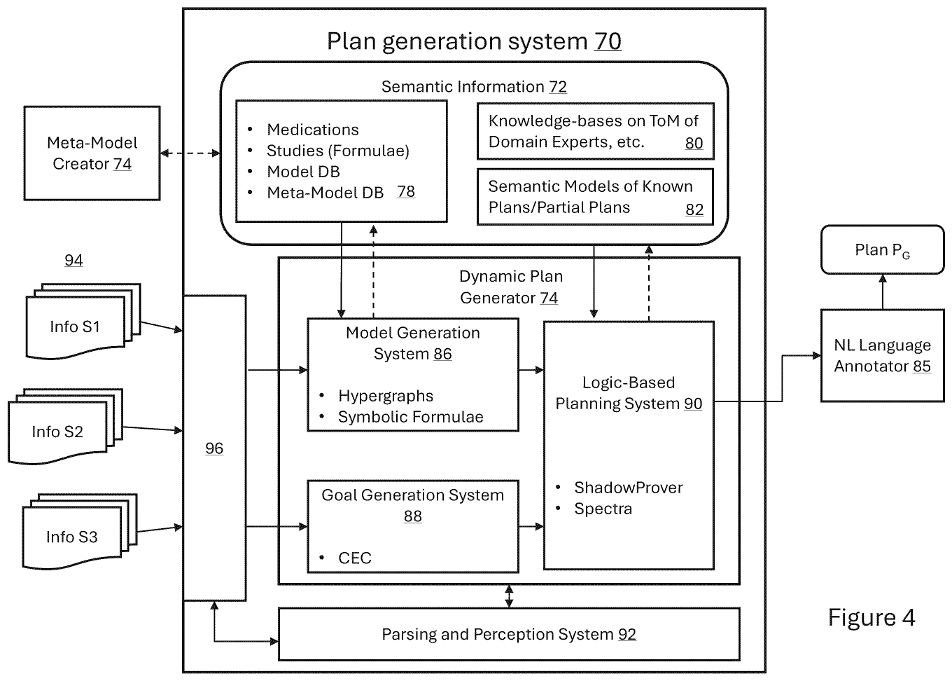

FIG. 4 depicts a plan generation system 70 that receives event information 94 from various sources S1, S2, S3 associated with dietary or emergency platform, and generates plans (PG) for humans (e.g., doctors, dieticians, emergency responders, and/or artificial agents. Event information may be collected from any type of input device, e.g., sensors, speakers, microphones, scanners, cameras, text inputs, computer systems, dispatch systems, etc. Platform 70 generally includes semantic information 72, a dynamic plan generator 74, and an information processor. Semantic information provides formalized knowledge of dietary or emergency response domains, which is used as the basis to create plans. Dynamic plan generator 74 is the engine that creates (and re-creates) models and plans for a current dietary plans or emergency events based on event information 94 and semantic information. Information processor is responsible for receiving event information 94, e.g., sensor data, inputs in a natural language (NL) form, and converting the event information 94 into a dedicated, highly expressive formal language for modeling and resolving health challenges and emergencies, denoted as L ERR. Information processor operates in conjunction with a parsing and perception system that continuously parses and evaluates incoming information 94 associated with user health or during an emergency event.

For the purposes of this disclosure, the term “event information 4094 refers to any data associated with health or a current emergency event, and may include structured and unstructured sensor, text, speech, image data, audio data, geospatial data, etc. Furthermore, it is understood that event information 94 may originate from any source, including smart toilet, human generated, computer generated, AI generated, sources. As health and/or event information 94 is received, all such data is translated into formulae at an appropriate level in a logically controlled natural language specifically tailored for dietary planning, as well as emergency response and rescue, referred to herein as L ERR. (Note that the event information 94 may also be saved in its original form for additional AI processing, such as using or training a large language model.) In some embodiments, the language L ERR is a sub-language of an enhanced version of a comprehensive, six-level, hierarchical formal Cognitive Calculus based language CC described in Patent No. U.S. Pat. No. 11,379,732, the contents of which is hereby incorporated by reference.

Referring again to FIG. 4, as the event information 94 flows into platform 70, it is translated by information processor into L ERR and fed to model generation system 86 and goal generation system 88. A model of the user's health may for example be created based on a meta-model selected from a meta-model database 78, as well as other semantic information 72 such as medical studies expressed as formulae. A generated model may for example be represented as a hypergraph GM and/or as symbolic formulae PM, and, e.g., details the actors involved, the type of health issues or emergency, available equipment and resources, etc., relationships, mental states, beliefs, knowledge, etc. Goal generation system 88 is responsible for generating a goal y, e.g., generate a dietary plan, call EMS, etc., which can be represented in cognitive event calculus (CEC), a logic-based formalism for representing actions and their effects.

Goal generation system 88 may for example be implemented as follows. The instant a sensor data is received and processed, parsing and perception system is activated, and while being assessed for intrinsic credibility, or a belief by the human fielding the notification that this is indeed a real emergency: select from the part of the ontology what type of diet should be deployed or emergency is believed to be transpiring. The goal then is simply to fulfill the dietary requirement or resolve that emergencies.

Note that after the model (and goal) are generated, they can be stored in a model database as part of the semantic information 72 for future use. As noted, meta-models, which are described in further detail herein, form the basis for creating models, i.e., they provide the underlying model structure for different emergency scenarios (e.g., car accident, search and rescue, fire, etc.). Meta-models may for example be created with a meta-model creator 24, e.g., using (1) manual engineering; or (2) automated induction applied to models, which can be created by generative AI applied to known prior models, suitably encoded.

Generating models with such inductive automated reasoning performed on content expressed in the formal language of the enhanced cognitive calculus can be done as follows. Namely, the enhanced calculi can be used to process a collection of N particular models given as input, and compile by inductive reasoning these into M meta-models (where M<<N). The meta-models hold information common to a subset of the N particular models in compact, instantiable fashion. Inductive reasoning here is achieved via various proof methods; generalization and anti-unification are two methods used for this purpose.

Once the model and goal are generated, they are forwarded to logic-based planning system 90 that generates a plan P. In one embodiment, planning system 90 receives the model both as hypergraph GM and a symbolic formulae PM, and a plan P is generated for both input types that, to a high level of probability (or likelihood) will secure that goal y given the model currently assumed. In one approach, logic-based planning system 90 is implemented using existing tools, Spectra™ and ShadowProver™, which utilize semantic information 72, namely knowledgebases on Theory-of-Mind (ToM) of domain experts, investigators, auditors, etc., and semantic models of known plans and partial plans. Spectra provides a mechanism for generating new plans based on an inputted goal, and ShadowProver provides a mechanism for logically testing plans to ensure they meet requirements of a response. As such, each resulting plan is evaluated as a proof to determine if it meets the goals of the response, e.g., ShadowProver will attempt to prove whether the response can be implemented as required, e.g., by domain experts and the like.

As noted, parsing and perception system 92 continuously analyzes event information as it is received and processed in platform 70 to ensure that a current model and/or plan are viable. From the initial input of event information 94 to final output of a plan, dataflow from each module to a next can be examined by parsing and perception system 92 to ensure viability. If a current model or plan is deemed no longer viable, parsing and perception system 92 may void the current approach, and cause a new model and/or a new plan to be created. A plan, model, argument, proof, proposition in a knowledge base, etc.; all such are defeasible, which entails that as new information arrives, any of these elements can be defeated (i.e., refuted, contradicted, supplanted with something more likely, etc.).

Evaluating a current model and or plan for viability may for example be accomplished with an automated reasoner (such as that detailed in the '732 patent), which operates over content expresses in the enhanced highly expressive 6-level language L ERR. In the case of analyzing a model for viability, one type of defeat of a model happens when new information that is of higher level of likelihood or probability literally contradicts one or more formulae in the model that is used as a premise. For example, assume there was an initial sensor reading that indicated the user had a medical condition, and then later multiple readings were received that the user was on a particular medication. The later reports may be assigned a higher score of cognitive-likelihood than the earlier report. This would potentially require a new model, e.g., one that involves the medication. The automated reasoner continuously runs to see if there is an inconsistency between new declarative information that is parsed and perceived into the platform 70, and the current, operative model. If an inconsistency is detected (where, again, the new information that leads to inconsistency is sufficiently likely/probably relative to that which is contradicts), the model generation system 86 is re-engaged to create a new model. Likewise, a new goal/plan may be required, which would be generated by goal generation system 88.

In the case of analyzing a current plan for viability, the automated reasoner may evaluate various conditions, e.g., preconditions for actions that are part of the operative plan no longer hold (e.g., the user cannot tolerate a type of food) and the current plan will fail. Postconditions (i.e., things which become true after actions in a plan are performed) may likewise be continuously checked, which were they to become true, would be logically inconsistent with any states-of-affairs/propositions that must not be violated. For example, a know side effect (stored as semantic information) would be broken. If such an inconsistency is detected, then planning system must be called to search for a different plan that is consistent with present knowledge and belief. A third condition that could trigger re-planning is if the automated reasoner is able to establish that there is some inconsistency “inside” an agent with what is newly parsed and perceived. In embodiments described herein, every agent has capabilities formally defined by functions that take percepts to actions, i.e., plans consist of actions to be performed. Accordingly, if some key capacity needed for performing one or more actions in a plan is lost, the automated reasoner will detect this inconsistency, and cause a new plan to be generated. A description of defeasible automated reasoning is for example described in Bringsjord, S., Giancola, M. & Govindarajulu, N. S. (2023), “Logic-Based Modeling of Cognition,”’ in Sun, R., ed., The Cambridge Handbook on Computational Cognitive Sciences (Cambridge, UK: Cambridge University Press), pp. 173-209. A preprint can be obtained via this link: http://kryten.mm.rpi.edu/SBringsjord_etal_L-BMC_121521.pdf, the contents of which is hereby incorporated by reference.

In the illustrative embodiment, plan PG is generated. PG may be output in any form suitable for human understanding, e.g., as a list of steps displayed on a tablet, audio instructions, augmented reality displays, etc. In some embodiments, PG is generated as a phased series of visual hypergraphs, viewable on display device, annotated with cogent expressions (e.g., in English), by language annotator 85. In still further embodiments, PG comprises a visual hypergraph annotated with spatial details, e.g., a directional compass rose, a map grid, landmarks, etc. P_{Φ} may be generated in any format suitable for an artificial agent. In other embodiments, maps are generated from the hypergraphs.

Referring again to FIG. 4, meta-model creator 74 is utilized to create meta-models of health situations and studies. Such studies are for example numerous All of these case studies can be used as raw material to manually create corresponding models, of both the symbolic, formula-based type, and the hypergraphical variety. With these models as input, abstract schemata for the particulars in these models can be automatically induced.

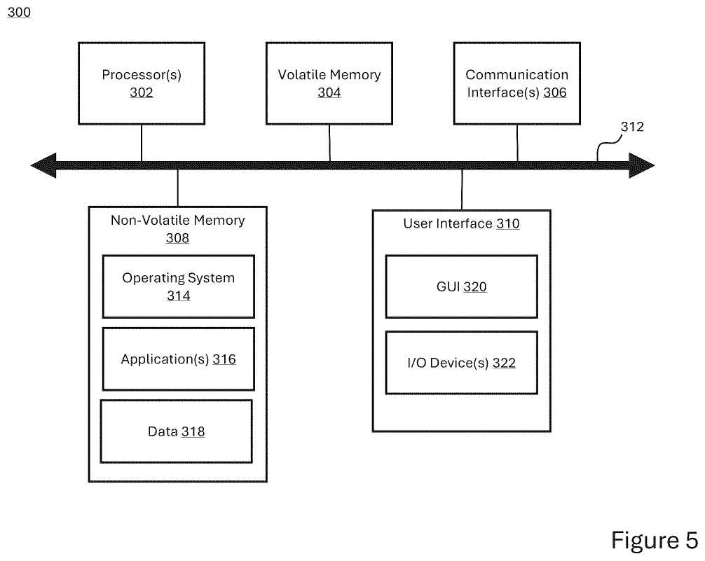

Elements of the described solution may be embodied in a computing system, such as that shown in FIG. 5 in which a computing device 300 may include one or more processors 302, volatile memory 304 (e.g., RAM), non-volatile memory 308 (e.g., one or more hard disk drives (HDDs) or other magnetic or optical storage media, one or more solid state drives (SSDs) such as a flash drive or other solid state storage media, one or more hybrid magnetic and solid state drives, and/or one or more virtual storage volumes, such as a cloud storage, or a combination of such physical storage volumes and virtual storage volumes or arrays thereof), user interface (UI) 310, one or more communications interfaces 306, and communication bus 312. User interface 310 may include graphical user interface (GUI) 320 (e.g., a touchscreen, a display, etc.) and one or more input/output (I/O) devices 322 (e.g., a mouse, a keyboard, etc.). Non-volatile memory 308 stores operating system 314, one or more applications 316, and data 318 such that, for example, computer instructions of operating system 314 and/or applications 316 are executed by processor(s) 302 out of volatile memory 304. Data may be entered using an input device of GUI 320 or received from I/O device(s) 322. Various elements of computer 300 may communicate via communication bus 312. Computer 300 as shown in FIG. 25 is shown merely as an example, as clients, servers and/or appliances and may be implemented by any computing or processing environment and with any type of machine or set of machines that may have suitable hardware and/or software capable of operating as described herein.

Processor(s) 302 may be implemented by one or more programmable processors executing one or more computer programs to perform the functions of the system. As used herein, the term “processor” describes an electronic circuit that performs a function, an operation, or a sequence of operations. The function, operation, or sequence of operations may be hard coded into the electronic circuit or soft coded by way of instructions held in a memory device. A “processor” may perform the function, operation, or sequence of operations using digital values or using analog signals. In some embodiments, the “processor” can be embodied in one or more application specific integrated circuits (ASICs), microprocessors, digital signal processors, microcontrollers, field programmable gate arrays (FPGAs), programmable logic arrays (PLAs), multi-core processors, or general-purpose computers with associated memory. The “processor” may be analog, digital or mixed-signal. In some embodiments, the “processor” may be one or more physical processors or one or more “virtual” (e.g., remotely located or “cloud”) processors.

Communications interfaces 306 may include one or more interfaces to enable computer 300 to access a computer network such as a LAN, a WAN, or the Internet through a variety of wired and/or wireless or cellular connections.

In described embodiments, a first computing device 300 may execute an application on behalf of a user of a client computing device (e.g., a client), may execute a virtual machine, which provides an execution session within which applications execute on behalf of a user or a client computing device (e.g., a client), such as a hosted desktop session, may execute a terminal services session to provide a hosted desktop environment, or may provide access to a computing environment including one or more of: one or more applications, one or more desktop applications, and one or more desktop sessions in which one or more applications may execute.

As will be appreciated by one of skill in the art upon reading the following disclosure, various aspects described herein may be embodied as a system, a device, a method or a computer program product (e.g., a non-transitory computer-readable medium having computer executable instruction for performing the noted operations or steps). Accordingly, those aspects may take the form of an entirely hardware embodiment, an entirely software embodiment, or an embodiment combining software and hardware aspects. Furthermore, such aspects may take the form of a computer program product stored by one or more computer-readable storage media having computer-readable program code, or instructions, embodied in or on the storage media. Any suitable computer readable storage media may be utilized, including hard disks, CD-ROMs, optical storage devices, magnetic storage devices, and/or any combination thereof.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. “Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise. “Approximately” as applied to a particular value of a range applies to both values, and unless otherwise dependent on the precision of the instrument measuring the value, may indicate +/−10% of the stated value(s).

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

The foregoing drawings show some of the processing associated according to several embodiments of this disclosure. In this regard, each drawing or block within a flow diagram of the drawings represents a process associated with embodiments of the method described. It should also be noted that in some alternative implementations, the acts noted in the drawings or blocks may occur out of the order noted in the figure or, for example, may in fact be executed substantially concurrently or in the reverse order, depending upon the act involved. Also, one of ordinary skill in the art will recognize that additional blocks that describe the processing may be added.

What is claimed is:

1. An intelligent toilet system, comprising:
a toilet having a waste analysis system, where the waste analysis system includes sensors that analyze microbiomes in human waste of a user;
an artificial intelligence (AI) system that receives sensor data from the waste analysis system and generates a health profile of the user; and
a hypergraphical reasoning platform that generates a dietary plan from the health profile and validates the plan using logics.

2. The intelligent toilet system of claim 1, wherein the AI system includes a model trained with a database of sensor values and health profiles.

3. The intelligent toilet system of claim 2, wherein the health profile includes a history of health profiles of the user.

4. The intelligent toilet system of claim 3, wherein the AI system generates alert conditions in response to a detected emergency condition of the user.

5. The intelligent toilet system of claim 4, wherein the hypergraphical platform generates an emergency response plan and validates the plan in response to the detected emergency condition.

6. The intelligent toilet system of claim 5, further comprising communication hardware for contacting emergency medical services based on the emergency response plan.

7. The intelligent toilet system of claim 1, wherein the plan is displayed on an output device as an annotated hypergraph.

8. The intelligent toilet system of claim 7, wherein the annotated hypergraph is periodically updated with new event information.

9. A method, comprising:
providing a toilet having a waste analysis system;
collecting human waste of a user in the waste analysis system;
analyzing microbiomes in the human waste and generating sensor data;
using an artificial intelligence (AI) system to process the sensor data and generate a health profile of the user; and
generating a dietary plan from the health profile and validating the dietary plan using a hypergraphical platform using logics.

10. The method of claim 9, wherein the AI system includes a model trained with a database of sensor values and health profiles.

11. The method of claim 10, wherein the health profile includes a history of health profiles of the user.

12. The method of claim 11, wherein the AI system generates alert conditions in response to a detected emergency condition of the user.

13. The method of claim 12, wherein the hypergraphical platform generates an emergency response plan and validates the plan in response to the detected emergency condition.

14. The method of claim 13, further comprising providing communication hardware for contacting emergency medical services based on the emergency response plan.

15. The method of claim 9, wherein the plan is displayed on an output device as an annotated hypergraph.

16. The method of claim 10, wherein the annotated hypergraph is periodically updated with new event information.
